(19) United States
(12) Patent Application Publication
(10) Pub. No.: US 2024/0000850 A1
(43) Pub. Date: Jan. 4, 2024

(54) METHODS AND COMPOSITIONS FOR ALLERGY AND ASTHMA TREATMENT USING FIBROBLASTS

(71) Applicant: FIGENE, LLC, Houston, TX (US)

(72) Inventor: Thomas ICHIM, San Diego, CA (US)

(21) Appl. No.: 17/907,115

(22) PCT Filed: Apr. 6, 2021
(86) PCT No.: PCT/US2021/025959

§ 371 Date: Sep. 23, 2022

Related U.S. Application Data
(60) Provisional application No. 63/006,957, filed on Apr. 8, 2020.

(51) Int. Cl. A61K 35/33; A61P 11/06; A61P 37/08; C12N 5/077

(52) U.S. Cl. CPC A61K 35/33; A61P 11/06; A61P 37/08; C12N 5/0656

(57) ABSTRACT

Disclosed herein are methods and compositions for reducing inflammation in a subject. Aspects are directed to reduction of pulmonary inflammation using fibroblasts or fibroblast derived products. In some cases, fibroblasts or fibroblast-derived products are provided to a subject to reduce pulmonary inflammation, thereby treating or preventing airway allergy, asthma, or asthma-related symptoms. Embodiments encompass use of fibroblast-derived products, including exosomes, conditioned media, and other components derived from fibroblasts.

This application claims priority to U.S. Provisional Patent Application Ser. No. 63/006,957, filed Apr. 8, 2020, which is incorporated by reference herein in its entirety.

### TECHNICAL FIELD

Embodiments of the disclosure encompass at least the fields of cell biology, molecular biology, immunology, and medicine.

### BACKGROUND

Asthma is a common disease condition in which inflammation develops in the airway and the lungs. It is estimated that between 235 and 330 million people suffer from asthma worldwide, leading to 250,000-345,000 deaths annually. Asthmatic patients often experience symptoms such as cough, wheezing, and shortness of breath, which considerably affect quality of life and may lead to complications and death.

From a cellular perspective, the pathology of asthma is characterized by accumulation and activation of immune cells in the airway mucosa, including eosinophils, neutrophils, macrophages, and T cells, which secrete a variety of immunological mediators. During the process of airway inflammation, naive CD4+T helper cells differentiate into type 2 helper T cells (Th2 cells), and secrete pro-inflammatory cytokines, such as interleukin (IL)-4, IL-5 and IL-13, which induce antibody production and eosinophil activation, further contributing to inflammation in the airway. While no cure exists for asthma, pharmaceutical drugs such as corticosteroids, bronchodilators, 02-adrenergic receptor agonists, anti-cholinergic drugs, and anti-leukotriene agents are usually used to reduce symptoms. Nevertheless, these drugs produce serious side effects and only provide temporary symptom relief.

Asthma may be considered an allergic reaction of the airway when symptoms occur in response to allergens such as hay or pollen. Allergies represent hypersensitive reactions of the immune system against substances found in the environment, including food, hay, pollen, insect stings, or pharmaceutical drugs. Acute allergies may lead to a serious condition called anaphylactic shock which may cause death. Allergies are characterized by binding of immunoglobulin E (IgE) antibodies to an allergen, followed by activation of basophils, secretion of histamine and other pro-inflammatory mediators, and induction of the inflammatory response. Prevention of airway allergies usually requires that the individual avoids allergens, air pollution, or cigarette smoking. Although many pharmaceutical drugs are available to treat symptoms of pulmonary allergies, for example, anti-histamines, epinephrine, glucocorticoids, and anti-leukotriene agents, these drugs only reduce the severity of symptoms and may produce adverse side effects.

Epidemiologic studies of longitudinal lung function over a 15-year period in adult asthmatics compared to non-asthmatics demonstrated that asthmatics had a greater decline in forced expiratory volume in 1 second (FEV1) over time than non-asthmatic controls. In one large study of 17,506 subjects, asthmatics had a greater decline in FEV1 (38 mL/year) than non-asthmatics (22 mL/year) (1). In addition, asthmatics who smoked tobacco had a greater decline in lung function than those who did not.

The cytokines associated with allergy can mediate upregulation of adhesion molecules and inflammatory chemokine production, and thereby immune-cell recruitment, degranulation of eosinophils, synthesis of IgE, and hyper-reactivity of smooth muscle. IL4 and IL-13 are structurally related molecules that share the common IL4Ra chain in receptor complexes. Although they exhibit overlapping function and both are associated with allergic disease, studies in IL4 deficient animals have demonstrated that IL-13 may be especially critical for the induction of AHR. IL-5 is central to eosinophil maturation, differentiation, activation and survival. The development of airway eosinophilia is associated with increased IL-5 expression in the airway mucosa and elevated concentrations of IL-5 in the luminal fluid and serum. Additionally, studies in mice have indicated the role of IL-5 in eosinophilia through depletion in murine models of asthma. Therefore Th2 mediated cytokines play an important role in generating the inflammation that characterizes allergic diseases.

Even patients with mild disease show airway inflammation, including infiltration of the mucosa and epithelium with activated T cells, mast cells, and eosinophils. T cells and mast cells release cytokines that promote eosinophil growth and maturation and the production of IgE antibodies, and these, in turn, increase microvascular permeability, disrupt the epithelium, and stimulate neural reflexes and mucus-secreting glands. The result is airway hyper-reactivity, bronchoconstriction, and hypersecretion, manifested by wheezing, coughing, and dyspnea.

### BRIEF SUMMARY

The present disclosure is directed to methods and compositions for reducing inflammation in a subject. Certain aspects are directed to the use of fibroblasts or fibroblast-derived products (e.g., exosomes, conditioned media, etc.) for treatment of asthma and other conditions associated with pulmonary inflammation. Fibroblasts or fibroblast-derived products may be used to treat asthma and/or to prevent asthma-related symptoms (i.e., prophylactic treatment). Aspects of the present disclosure demonstrate that fibroblasts and fibroblast-derived products (e.g., conditioned media from fibroblasts) significantly reduce the symptoms of asthma and airway allergies in mammals, without producing significant toxic side effects. Accordingly, embodiments of the disclosure provide a new strategy to treat asthma and airway allergies.

Disclosed herein, in some embodiments, is a method of treating or preventing asthma in a subject comprising providing to the subject an effective amount of fibroblasts or fibroblast-derived products. In some embodiments, the method comprises providing to the subject an effective amount of fibroblasts. In some embodiments, the fibroblasts are plastic adherent. In some embodiments, the fibroblasts express CD105. In some embodiments, the fibroblasts express CD73. In some embodiments, the fibroblasts are xenogenic. In some embodiments, the fibroblasts are allogenic. In some embodiments, the fibroblasts are autologous. In some embodiments, the method further comprises, prior to providing to the subject the effective amount of fibroblasts, exposing the fibroblasts to oxytocin. In some embodiments, the fibroblasts are exposed to oxytocin at a concentration of between 1 and 100 IU per ml.

In some embodiments, the method comprises providing to the subject an effective amount of fibroblast-derived products. In some embodiments, the fibroblast-derived products comprise conditioned media derived from fibroblasts. In some embodiments, the conditioned media is capable of stimulating proliferation of CD8+ cells in the subject. In some embodiments, the conditioned media comprises FGF-1. In some embodiments, the conditioned media comprises at least 2 ng/ml of FGF-1. In some embodiments, the conditioned media comprises FGF-2. In some embodiments, the conditioned media comprises at least 5 ng/ml of FGF-2. In some embodiments, the conditioned media comprises TGF-β. In some embodiments, the conditioned media comprises at least 20 pg/ml of TGF-β. In some embodiments, the fibroblast-derived products comprise microvesicles from fibroblasts. In some embodiments, the fibroblast-derived products comprise exosomes from fibroblasts. In some embodiments, the exosomes comprise CD81. In some embodiments, the exosomes comprise TNF-β. In some embodiments, the exosomes are between 60 and 200 nm in size. In some embodiments, the fibroblast-derived products comprise apoptotic vesicles from fibroblasts. the fibroblast-derived products comprise nucleic acids from fibroblasts.

In some embodiments, the method comprises reducing pulmonary inflammation in the subject. In some embodiments, the pulmonary inflammation is associated with exposure to an allergen. In some embodiments, the fibroblasts or fibroblast-derived products are capable of inhibiting eosinophil activation in the subject. In some embodiments, the fibroblasts or fibroblast-derived products are capable of inhibiting basophil activation in the subject. In some embodiments, the fibroblasts or fibroblast-derived products are capable of inhibiting mast cell activation in the subject.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIGS. 1A and 1B show results from experiments described in Example 1. Shown from left to right for each time period are results from 1) control conditions, 2) dead fibroblast conditions, 3) live fibroblast conditions, and 4) conditioned media conditions. FIG. 1A shows sneeze counts from mice following intranasal challenge. FIG. 1B shows nasal rubbing movement counts from mice following intranasal challenge.

FIG. 2 shows eosinophil counts in the nasal septum from animals treated in the experiments described in Example 2. Shown from left to right for each time period are results from 1) control conditions, 2) dead fibroblast conditions, 3) live fibroblast conditions, and 4) conditioned media conditions.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

“Autologous,” as used herein, refers to cells derived from the same subject and/or having the same genetic material.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic bodies, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

As used herein, a “trophic factor” describes a substance that promotes and/or supports survival, growth, proliferation and/or maturation of a cell. Alternatively or in addition, a trophic factor stimulates increased activity of a cell.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

### II. Methods and Compositions for Disease Treatment or Prevention

Aspects of the present disclosure are directed to methods and compositions for reducing inflammation in a subject (e.g., pulmonary inflammation) for treatment or prevention of inflammatory conditions (e.g., airway allergy and asthma). In some embodiments, methods comprise administering an effective amount of fibroblasts and/or fibroblast-derived products to a subject, thereby reducing pulmonary inflammation and treating or preventing asthma. In some embodiments, between 10,000 and 10 million cells per kg are administered to the subject. In some embodiments, about 1 million cells per kg are administered to the subject. In some embodiments, methods comprise administering fibroblast-derived products to a subject, thereby reducing pulmonary inflammation and treating or preventing asthma. Fibroblasts or fibroblast-derived products may be used to significantly reduce secretion of one or more pro-inflammatory cytokines (e.g., IL-4, IL-5, IL-13), reduce airway hyper-responsiveness, reduce eosinophil accumulation, reduce mast cell degranulation, and/or reduce production of allergen-specific antibodies (e.g., IgG, IgE). In specific embodiments, a subject's asthma is delayed in onset and/or frequency and/or severity or is completely prevented. A subject may have a reduced risk of having an asthma attack, having a severe asthma attack, or dying when utilizing methods and compositions of the disclosure.

Embodiments of the disclosure are directed to a method of treating or preventing asthma in a subject comprising providing to the subject an effective amount of fibroblasts and/or fibroblast-derived products. Fibroblasts used herein may be plastic adherent fibroblasts. Fibroblasts may express various markers, including, e.g., CD105 and/or CD73, as disclosed further elsewhere herein. Fibroblasts may be xenogenic, allogenic, autologous fibroblasts, or a mixture thereof. In some embodiments, fibroblasts are exposed to an effective amount of oxytocin (e.g., at a concentration of between 1 and 100 IU per ml) prior to providing them to a subject. Fibroblast-derived products used herein may include conditioned media from fibroblasts. Conditioned media may comprise various components, including FGF-1 (e.g., at least 2 ng/ml), FGF-2 (e.g., at least 5 ng/ml), and/or TGF-β (at least 20 pg/ml). In some embodiments, fibroblast-derived products comprise microvesicles or exosomes from fibroblasts. In some embodiments, microvesicles or exosomes used in the disclosed methods comprise CD81 and/or TNF-β. In some embodiments, fibroblast-derived products comprise apoptotic vesicles. In some embodiments, fibroblast-derived products comprise nucleic acids (e.g., mRNA, miRNA, etc.). Providing fibroblasts and/or fibroblast-derived products to a subject may serve to reduce pulmonary inflammation (e.g., inflammation associated with exposure to an allergen) in the subject. In some embodiments, the fibroblasts or fibroblast-derived products are capable of inhibiting activation of inflammatory cells including, for example, eosinophils, basophils, and/or mast cells in the subject.

In some embodiments, disclosed herein are methods for activating NF-κB signaling in fibroblasts as a mechanism for enhancing the ability of fibroblasts to suppress allergic inflammation.

In some embodiments, disclosed are methods comprising activation of fibroblasts prior to providing fibroblasts to a subject (e.g., for therapeutic use). Activation of fibroblasts may comprise, for example, administration of oxytocin. In one example, fibroblasts are cultured with oxytocin at a concentration of between 1 and 100 IU/ml (e.g., about 10 IU/ml) to generate activated fibroblasts; the activated fibroblasts are then provided to a subject to reduce or prevent pulmonary inflammation.

Methods of the present disclosure include methods for treating or preventing an asthma-related condition. Examples of asthma-related conditions which can be treated using compositions and methods of the present disclosure include bronchial inflammation, excess bronchial mucus or plugs, lung tissue damage, eosinophil accumulation, bronchospasm, narrowing of breathing airways, airway hypersensitivity, airway remodeling, wheezing, breathlessness, chest tightness, coughing, dyspnea, burning, airway edema, tachypnea, tachycardia, cyanosis, allergic rhinitis, infections (e.g., fungal or bacterial), atopic dermatitis, biorhythm abnormalities, Churg-Strauss syndrome, and gastroesophageal reflux disease. One or more symptoms of one or more of these asthma-related conditions may be improved or eliminated with methods and compositions of the disclosure.

In some embodiments, fibroblasts and/or fibroblast-derived products are provided to a subject together with a bronchodilator. In some embodiments, a bronchodilator is a (32 agonist. In some embodiments, a bronchodilator is a methylxanthine. The bronchodilator may be given before, after, or both in relation to the fibroblasts and/or fibroblast-derived products.

In some embodiments, methods and compositions of the present disclosure comprise fibroblasts and/or fibroblast-derived products together with one or more asthma therapies. Asthma therapies include, for example, beta-2 agonists, anticholinergics, corticosteroids, glucocorticosteroids, anti-allergenic s, anti-inflammatories, bronchiodialators, expectorants, allergy medications, cromolyn sodium, albuterol, beclomethasone dipropionate inhaler, budesonide inhaler, fluticasone and salmeterol oral inhaler, fluticasone propionate oral inhaler, hydrocortisone oral, ipratropium bromide inhaler, montelukast, prednisone, salmeterol, terbutaline, theophylline, triamcinolone acetonide inhaler, methotrexate (MTX); interleukin antagonists such as IL-4, IL-5, IL-12 antibodies, receptor proteins or antagonists, and antagonist fusion proteins, IgE antibodies and antagonists, CD4 antagonists, antileukotrienes, platlet activating factor, thromoboxane antagonists, tryptase inhibitors, NK2 receptor antagonists, ipratropium, thephyllene, disodium chromoglycate (DSCG), functional or structural analogs thereof, and derivatives or variants thereof. The therapies may be given before, after, or both in relation to the fibroblasts and/or fibroblast-derived products.

In some embodiments, methods and compositions of the present disclosure comprise fibroblasts and/or fibroblast-derived products together with one or more additional agents. In some embodiments, the additional agent is an asthma-related therapeutic, a TNF antagonist (e.g., TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline), an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine, and/or a cytokine antagonist.

In some embodiments, fibroblasts and/or fibroblast-derived products are provided together with one or more anti-leukotrienes. Anti-leukotrienes are members of a heterogeneous class of anti-asthma agents with the potential to interfere with the initial steps in the inflammatory cascade. Leukotrienes are inflammatory substances related to prostaglandins; both are generated from arachidonic acid in cell membranes. After arachidonic acid in mast cells, eosinophils, macrophages, monocytes, and basophils is formed, it is metabolized via two major pathways: (1) a cycloxygenase pathway (which produces prostaglandins and thromboxanes) and (2) the 5-lipoxygenase pathway, which produces leukotrienes in the cytoplasma. The leukotrienes are well known as the slow reacting substance of anaphylaxis (“SRS-A”). Leukotrienes play an important role in bronchial inflammation. They induce migration, adhesion and aggregation of various white blood cells (e.g., neutrophils, eosinophils, and monocytes) to blood vessels, increase capillary permeability, and cause bronchial and vessel smooth muscle constriction.

### III. Fibroblasts and Cultured Cells

Aspects of the present disclosure comprise cells useful in therapeutic methods and compositions. Cells disclosed herein include, for example, fibroblasts, stem cells (e.g., hematopoietic stem cells and/or mesenchymal stem cells), and/or endothelial progenitor cells. Cells of a given type (e.g., fibroblasts) may be used alone or in combination with cells of other types. For example, fibroblasts may be isolated and provided to a subject alone or in combination with one or more stem cells. In some embodiments, disclosed herein are fibroblasts capable of reducing or preventing pulmonary inflammation. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of CD73, CD90, CD105, Oct-4, Nanog, Sox-2, KLF4, c-Myc, Rex-1, GDF-3, LIF receptor, CD105, CD117, CD344, Stella, telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, VEGF, CD10, CD13, CD44, CD73, CD90, CD141, PDGFr-α, HLA-A, HLA-B, and/or HLA-C markers.

In some embodiments, an isolated fibroblast cell does not express at least one of MHC class I, MHC class II, CD45, CD13, CD49c, CD66b, CD73, CD105, CD90, CD31, CD34, CD117, CD141, HLA-DR, HLA-DP, and/or HLA-DQ. Such isolated fibroblast cells may be used as a source of conditioned media. The cells may be cultured alone, or may by cultured in the presence of other cells in order to further upregulate production of growth factors in the conditioned media.

Fibroblasts may be expanded and utilized by administration themselves, or may be cultured in a growth media in order to obtain conditioned media. The term Growth Medium generally refers to a medium sufficient for the culturing of fibroblasts. In particular, one example of a medium for the culturing of the cells of the disclosure herein comprises Dulbecco's Modified Essential Media (DMEM). In particular embodiments it is DMEM-low glucose (also DMEM-LG herein) (Invitrogen®, Carlsbad, Calif.). The DMEM-low glucose may be supplemented with 15% (v/v) fetal bovine serum (e.g. defined fetal bovine serum, Hyclone™, Logan Utah), antibiotics/antimycotics (such as penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods that derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and a particular marker expression pattern is determined.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote angiogenesis. An “angiogenic gene” describes a gene encoding for a protein or polypeptide capable of stimulating or enhancing angiogenesis in a culture system, tissue, or organism. Examples of angiogenic genes that may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TGF-β, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-β, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secrete one or more factors prior to or following introduction into an individual. Such factors include, but are not limited to, growth factors, trophic factors, and/or cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500 g). Conditioned media may be filtered through a membrane. The membrane may be a >1000 kDa membrane. Conditioned media may be subject to liquid chromatography such as HPLC. Conditioned media may be separated by size exclusion.

An example procedure for collecting dermal fibroblasts for use in the methods and compositions for the present disclosure is provided. In one embodiment, the starting material is composed of 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, and cells are pelleted by centrifugation and resuspended in 5.0 ml IMDM. Alternatively, centrifugation is not performed, with full inactivation of the enzyme occurring by the addition of IMDM only. IMDM is added prior to seeding of the cell suspension into a T-175 cell culture flask for initiation of cell growth and expansion. A T-75, T-150, T-185 or T-225 flask can be used in place of the T-75 flask. Cells are incubated at 37±2° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells may remain in the T-175 flask less than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37±2° C. with 5.0±1.0% CO₂ and passaged every three to five days in the T-500 flask and every five to seven days in the ten layer cell stack (10CS). Cells may remain in the T-500 flask no longer than 10 days prior to passaging. Quality Control (QC) release testing for safety of the Bulk Drug Substance includes sterility and endotoxin testing. When cell confluence in the T-500 flask is at least 95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2° C. with 5.0±1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium. When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

### IV. Exosomes

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing asthma or pulmonary inflammation. The microparticle may comprise (such as express) a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

Described herein are methods for obtaining and using exosomes and other membrane vesicles from fibroblasts. Example methods for purifying exosomes are provided.

In one embodiment, a strong or weak anion exchange is performed. In some embodiments, the anion exchange is performed via high performance liquid chromatography (HPLC). Different types of supports may be used to perform the anion exchange chromatography. Example supports include cellulose, poly(styrene-divinylbenzene), agarose, dextran, acrylamide, silica, ethylene glycol-methacrylate co-polymer, or mixtures thereof, e.g., agarose-dextran mixtures. In some embodiments, the present disclosure relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures. Supports used in anion exchange may be functionalized.

In some embodiments, to improve the chromatographic resolution, supports are used in bead form. Beads may have a homogeneous and calibrated diameter, with a sufficiently high porosity to enable the penetration of exosomes under chromatography. In this way, given the diameter of exosomes (generally between 50 and 100 nm) some embodiments comprise the use of high porosity gels, for example between 10 nm and 5 μm, between 20 nm and 2 μm, or between 100 nm and 1 μm. For the anion exchange chromatography, the support used may be functionalized using a group capable of interacting with an anionic molecule. This group may be composed of an amine which may be ternary or quaternary, which defines a weak or strong anion exchanger, respectively. In some embodiments, a strong anion exchanger is used. In some embodiments, the support is poly(styrene-divinylbenzene), acrylamide, agarose, dextran or silica, alone or in mixtures, and functionalized with a quaternary amine.

An example support for performing anion exchange chromatography comprises poly(styrene-divinylbenzene). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, for example using the passage of a saline solution gradient of increasing concentration, e.g. from 0 to 2 M. A sodium chloride solution may be used. The different fractions purified in this way may be detected by measuring their optical density (OD) at the column outlet using a continuous spectro-photometric reading.

Different types of columns may be used to perform the chromatographic step, according to requirements and the volumes to be treated. For example, depending on the preparations, it is possible to use a column from approximately 100 μl up to 10 ml or greater. It is understood that higher volumes may also be treated, by increasing the volume of the column, for example. In addition, it is also possible to combine the anion exchange chromatography step with a gel permeation chromatography step. In this way, according to a specific embodiment of the invention, a gel permeation chromatography step is added to the anion exchange step, either before or after the anion exchange chromatography step. In one embodiment, the permeation chromatography step takes place after the anion exchange step. In one embodiment, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. The process according to the invention may be applied to different biological samples. For example, these may consist of a biological fluid from a subject (bone marrow, peripheral blood, etc.), a culture supernatant, a cell lysate, a pre-purified solution or any other composition comprising membrane vesicles.

Exosome enrichment may comprise one or more centrifugation, clarification, ultrafiltration, nanofiltration, and/or affinity chromatography steps. In some embodiments, an enrichment comprises (i) the elimination of cells and/or cell debris (clarification), possibly followed by (ii) a concentration and/or affinity chromatography step. In some embodiments, an enrichment comprises an affinity chromatography step, in some cases preceded by a step of elimination of cells and/or cell debris (clarification). In some embodiments, an enrichment comprises (i) the elimination of cells and/or cell debris (clarification), (ii) a concentration, and (iii) an affinity chromatography. The cells and/or cell debris may be eliminated by centrifugation of the sample, for example, at a low speed (e.g., below 1000 g, between 100 and 700 g). Centrifugation conditions may comprise, for example, 300 g or 600 g for a period between 1 and 15 minutes.

Cells and/or cell debris may also be eliminated by filtration of the sample, possibly combined with the centrifugation described above. The filtration may particularly be performed with successive filtrations using filters with a decreasing porosity. For example, filters with a porosity above 0.2 μm, e.g. between 0.2 and 10 μm, may be used. It is particularly possible to use a succession of filters with a porosity of 10 μm, 1 μm, and 0.5 μm followed by 0.22 μm.

A concentration step may be performed, in order to reduce the volumes of sample to be treated during the chromatography stages. Concentration may be obtained by centrifugation of a sample at high speeds, e.g. between 10,000 and 100,000 g, to cause the sedimentation of membrane vesicles. This may comprise a series of differential centrifugations, with the last centrifugation performed at approximately 70,000 g. The membrane vesicles in the pellet obtained may be taken up with a smaller volume and in a suitable buffer for the subsequent steps of the process. The concentration step may be performed by ultrafiltration. Ultrafiltration may allow both to concentrate the supernatant and perform an initial purification of the vesicles. In some embodiments, the biological sample (e.g., a supernatant) is subjected to an ultrafiltration, e.g., a tangential ultrafiltration. Tangential ultrafiltration comprises concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes, flat membranes, or hollow fibers. In some embodiments, membranes have a cut-off threshold of below 1000 kDa, between 300 kDa and 1000 kDa, or between 300 kDa and 500 kDa.

Affinity chromatography can be performed in various ways, using different chromatographic support and material. For example, non-specific affinity chromatography may be used, aimed at retaining (i.e., binding) certain contaminants present within the solution, without retaining the objects of interest (e.g., exosomes). In some embodiments, an affinity chromatography on a dye is used, allowing the elimination (i.e., the retention) of contaminants such as proteins and enzymes, for instance albumin, kinases, dehydrogenases, clotting factors, interferons, lipoproteins, co-factors, etc. In some embodiments, the support used for this chromatography step is a support as used for ion exchange chromatography, functionalised with a dye.

In some embodiments, preparing membrane vesicles (e.g., exosomes) comprises a) the culture of a population of membrane vesicle-producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In some embodiments, step b) comprises a filtration of the culture supernatant followed by ultrafiltration (e.g., tangential ultrafiltration). In some embodiments, step b) comprises a clarification of the culture supernatant followed by an affinitiy chromatography on dye (e.g., Blue SEPHAROSE®).

The material harvested may, in some cases, be subjected to one or more additional treatment and/or filtration stages, particularly for sterilization purposes. For this filtration treatment stage, filters with a diameter less than or equal to 0.3 μm (e.g., 0.22 μm) may be used. After step d), the material obtained is, for example, distributed into suitable devices such as bottles, tubes, bags, syringes, etc., in a suitable storage medium. Purified vesicles obtained in this way may be stored cold, frozen, or used extemporaneously. An example preparation process comprises at least the following steps: c) an anion exchange chromatography and/or gel permeation chromatography treatment of the biological sample, and d) a filtration step, such as sterilizing filtration, of the material harvested after step c). In some embodiments, the process comprises c) an anion exchange chromatography treatment of the biological sample, and d) a filtration step, such as sterilizing filtration, on the material harvested after step c). In some embodiments, the process comprises: c) a gel permeation chromatography treatment of the biological sample, and d) a filtration step, such as sterilizing filtration, on the material harvested after step c). In some embodiments, the process comprises: c) an anionic exchange treatment of the biological sample followed or preceded by gel permeation chromatography, and d) a filtration step, such as sterilizing filtration, on the material harvested after step c).

### V. Administration of Therapeutic Compositions

The therapy provided herein may comprise administration of a therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about (or at least or no more than) 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 1-7 consecutive days. In some embodiments, 1-7 administrations are provided over the course of 1-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Fibroblasts Reduce Sneezing and Nasal Rubbing in an In Vivo Model of Asthma

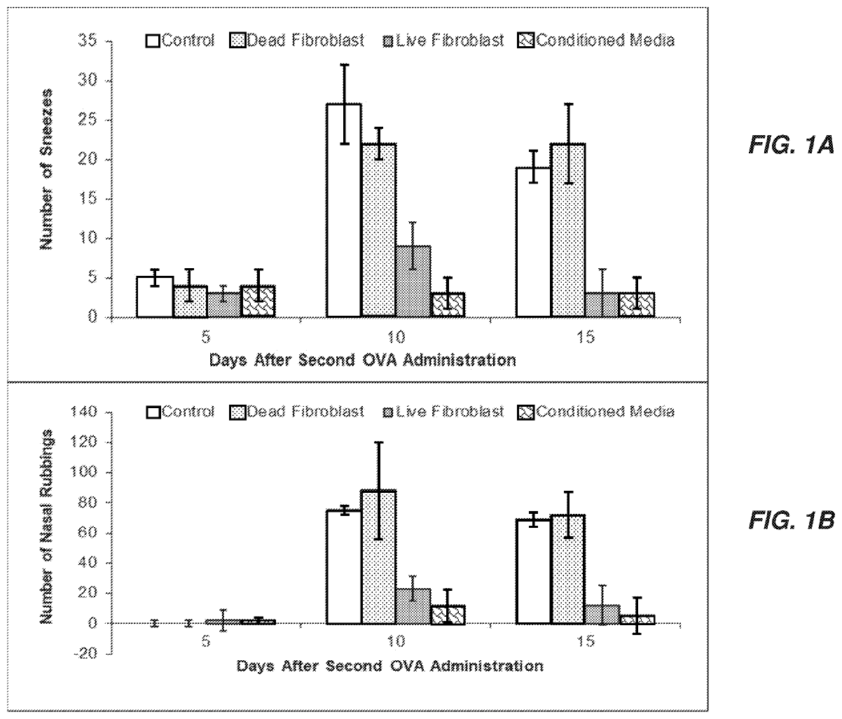

Six to eight week old male BALB/c mice were injected intravenously with 500,000 fibroblasts, 50 μg of fibroblast conditioned media, or saline twice on days 0 and 14. For generation of non-viable (“Dead”) fibroblasts, cells were subjected to three freeze-thaw cycles in liquid nitrogen, followed by sonication for five minutes. Mice were also injected i.p. with 10 μg of ovalbumin (OVA) and 4 mg of Al(OH₃) twice on days 2 and 16. Each group consisted of six mice. The mice were challenged intranasally at 5, 10, and 15 days after the last OVA injection, and the number of sneezing (FIG. 1A) and nasal rubbing movements (FIG. 1B) were counted for 20 minutes after intranasal challenge. The results demonstrate a reduction in allergic response in the groups treated with live fibroblasts or conditioned media from fibroblasts.

### Example 2: Fibroblast Reduce Eosinophilic Infiltration in an In Vivo Model of Asthma

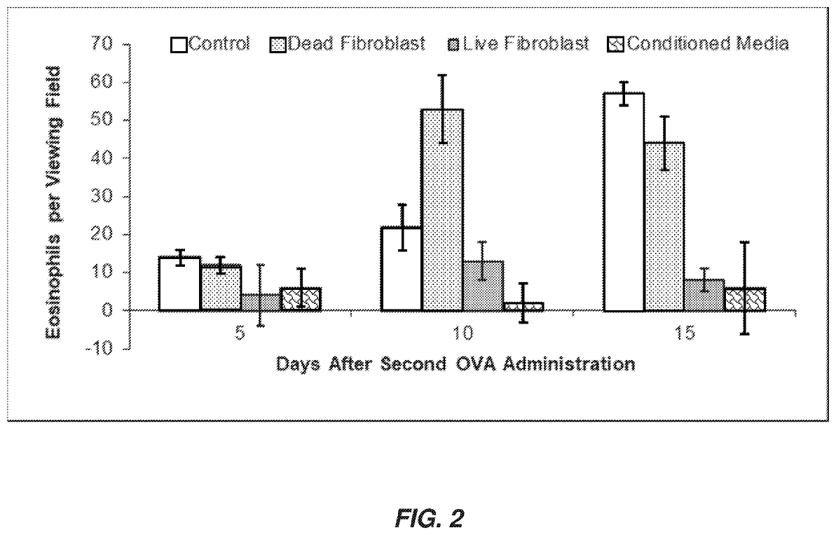

Allergic responses were generated and mice were treated as described in Example 1. A number of eosinophils in the nasal septum was determined for each mouse, the results of which are shown in FIG. 2. These results further demonstrate a reduction in allergic response in the groups treated with live fibroblasts or conditioned media from fibroblasts.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Lange, P., Parner, J., Vestbo, J., Schnohr, P., and Jensen, G. (1998) A 15-year follow-up study of ventilatory function in adults with asthma. N Engl J Med 339, 1194-1200
2. Oldham, P. D. (1987) Decline of FEV1. Thorax 42, 161-164
3. McGeachie, M. J., Yates, K. P., Zhou, X., Guo, F., Sternberg, A. L., Van Natta, M. L., Wise, R. A., Szefler, S. J., Sharma, S., Kho, A. T., Cho, M. H., Croteau-Chonka, D. C., Castaldi, P. J., Jain, G., Sanyal, A., Zhan, Y., Lajoie, B. R., Dekker, J., Stamatoyannopoulos, J., Covar, R. A., Zeiger, R. S., Adkinson, N. F., Williams, P. V., Kelly, H. W., Grasemann, H., Vonk, J. M., Koppelman, G. H., Postma, D. S., Raby, B. A., Houston, I., Lu, Q., Fuhlbrigge, A. L., Tantisira, K. G., Silverman, E. K., Tonascia, J., Weiss, S. T., and Strunk, R. C. (2016) Patterns of Growth and Decline in Lung Function in Persistent Childhood Asthma. N Engl J Med 374, 1842-1852
4. Hayden, L. P., Cho, M. H., Raby, B. A., Beaty, T. H., Silverman, E. K., Hersh, C. P., and Investigators, C. O. (2018) Childhood asthma is associated with COPD and known asthma variants in COPDGene: a genome-wide association study. Respir Res 19, 209
5. Frew, A. J., Corrigan, C. J., Maestrelli, P., Tsai, J. J., Kurihara, K., O'Hehir, R. E., Hartnell, A., Cromwell, O., and Kay, A. B. (1989) T lymphocytes in allergen-induced late-phase reactions and asthma. Int Arch Allergy Appl Immunol 88, 63-67
6. Kawano, Y., Noma, T., Maeda, K., and Yata, J. (1992) CD4+CD45RA+ T cells modulate allergen-induced interleukin 2 responsiveness in human lymphocytes. Clin Immunol Immunopathol 62, 327-335
7. Gratziou, C., Carroll, M., Walls, A., Howarth, P. H., and Holgate, S. T. (1992) Early changes in T lymphocytes recovered by bronchoalveolar lavage after local allergen challenge of asthmatic airways. Am Rev Respir Dis 145, 1259-1264
8. Robinson, D. S., Hamid, Q., Jacobson, M., Ying, S., Kay, A. B., and Durham, S. R. (1993) Evidence for Th2-type T helper cell control of allergic disease in vivo. Springer Semin Immunopathol 15, 17-27
9. Bellini, A., Yoshimura, H., Vittori, E., Marini, M., and Mattoli, S. (1993) Bronchial epithelial cells of patients with asthma release chemoattractant factors for T lymphocytes. J Allergy Clin Immunol 92, 412-424
10. Ricci, M., Matucci, A., and Rossi, 0. (1994) T cells, cytokines, IgE and allergic airways inflammation. J Investig Allergol Clin Immunol 4, 214-220
11. Anderson, G. P., and Coyle, A. J. (1994) TH2 and ‘TH2-like’ cells in allergy and asthma: pharmacological perspectives. Trends Pharmacol Sci 15, 324-332
12. Walker, C., Bauer, W., Braun, R. K., Menz, G., Braun, P., Schwarz, F., Hansel, T. T., and Villiger, B. (1994) Activated T cells and cytokines in bronchoalveolar lavages from patients with various lung diseases associated with eosinophilia. Am J Respir Crit Care Med 150, 1038-1048
13. Coyle, A. J., and Tsuyuki, S. (1995) Th2 cells and cytokine networks in allergic inflammation of the lung. Mediators Inflamm 4, 239-247
14. Gavett, S. H., O'Hearn, D. J., Li, X., Huang, S. K., Finkelman, F. D., and Wills-Karp, M. (1995) Interleukin 12 inhibits antigen-induced airway hyperresponsiveness, inflammation, and Th2 cytokine expression in mice. J Exp Med 182, 1527-1536
15. Erb, K. J., and Le Gros, G. (1996) The role of Th2 type CD4+ T cells and Th2 type CD8+ T cells in asthma. Immunol Cell Biol 74, 206-208
16. Krouwels, F. H., Hol, B. E., Bruinier, B., Lutter, R., Jansen, H. M., and Out, T. A. (1996) Cytokine production by T-cell clones from bronchoalveolar lavage fluid of patients with asthma and healthy subjects. Eur Respir J Suppl 22, 95s-103s
17. Krinzman, S. J., De Sanctis, G. T., Cernadas, M., Kobzik, L., Listman, J. A., Christiani, D. C., Perkins, D. L., and Finn, P. W. (1996) T cell activation in a murine model of asthma. Am J Physiol 271, L476-483
18. Umetsu, D. T., and DeKruyff, R. H. (1997) Th1 and Th2 CD4+ cells in the pathogenesis of allergic diseases. Proc Soc Exp Biol Med 215, 11-20
19. Brusselle, G. G., Kips, J. C., Tavernier, J. H., van der Heyden, J. G., Cuvelier, C. A., Pauwels, R. A., and Bluethmann, H. (1994) Attenuation of allergic airway inflammation in IL-4 deficient mice. Clin Exp Allergy 24, 73-80
20. Robinson, D., Hamid, Q., Ying, S., Bentley, A., Assoufi, B., Durham, S., and Kay, A. B. (1993) Prednisolone treatment in asthma is associated with modulation of bronchoalveolar lavage cell interleukin-4, interleukin-5, and interferon-gamma cytokine gene expression. Am Rev Respir Dis 148, 401-406
21. Humbert, M., Durham, S. R., Ying, S., Kimmitt, P., Barkans, J., Assoufi, B., Pfister, R., Menz, G., Robinson, D. S., Kay, A. B., and Corrigan, C. J. (1996) IL-4 and IL-5 mRNA and protein in bronchial biopsies from patients with atopic and nonatopic asthma: evidence against “intrinsic” asthma being a distinct immunopathologic entity. Am J Respir Crit Care Med 154, 1497-1504
22. Moverare, R., Rak, S., and Elfman, L. (1998) Allergen-specific increase in interleukin (IL)-4 and IL-5 secretion from peripheral blood mononuclear cells during birch-pollen immunotherapy. Allergy 53, 275-281
23. Borish, L. C., Nelson, H. S., Lanz, M. J., Claussen, L., Whitmore, J. B., Agosti, J. M., and Garrison, L. (1999) Interleukin-4 receptor in moderate atopic asthma. A phase I/II randomized, placebo-controlled trial. Am J Respir Crit Care Med 160, 1816-1823
24. Endo, H., Iwamoto, I., Nakajima, H., and Yoshida, S. (1993) In vitro interleukin-5 production of peripheral blood mononuclear cells is increased in patients with asthma. Int Arch Allergy Immunol 101, 425-430
25. Kotsimbos, A. T., and Hamid, Q. (1997) IL-5 and IL-5 receptor in asthma. Mem Inst Oswaldo Cruz 92 Suppl 2, 75-91
26. Foster, P. S., Hogan, S. P., Matthaei, K. I., and Young, I. G. (1997) Interleukin-4 and interleukin-5 as targets for the inhibition of eosinophilic inflammation and allergic airways hyperreactivity. Mem Inst Oswaldo Cruz 92 Suppl 2, 55-61
27. Till, S., Dickason, R., Huston, D., Humbert, M., Robinson, D., Larche, M., Durham, S., Kay, A. B., and Corrigan, C. (1997) IL-5 secretion by allergen-stimulated CD4+ T cells in primary culture: relationship to expression of allergic disease. J Allergy Clin Immunol 99, 563-569
28. Levy, F., Kristofic, C., Heusser, C., and Brinkmann, V. (1997) Role of IL-13 in CD4 T cell-dependent IgE production in atopy. Int Arch Allergy Immunol 112, 49-58
29. Wahlstrom, J., Dahlen, B., Ihre, E., Wigzell, H., Grunewald, J., and Eklund, A. (1998) Selective CD8+ T cells accumulate in the lungs of patients with allergic asthma after allergen bronchoprovocation. Clin Exp Immunol 112, 1-9
30. Pfeffer, P. E., Ho, T. R., Mann, E. H., Kelly, F. J., Sehlstedt, M., Pourazar, J., Dove, R. E., Sandstrom, T., Mudway, I. S., and Hawrylowicz, C. M. (2018) Urban particulate matter stimulation of human dendritic cells enhances priming of naive CD8 T lymphocytes. Immunology 153, 502-512
31. Zuska-Prot, M., and Maslanka, T. (2017) Inhaled glucocorticoid treatment prevents the response of CD8(+) T cells in a mouse model of allergic asthma and causes their depletion outside the respiratory system. Int Immunopharmacol 53, 63-72
32. Bruggemann, T. R., Fernandes, P., Oliveira, L. M., Sato, M. N., Martins, M. A., and Arantes-Costa, F. M. (2017) Cigarette Smoke Increases CD8alpha(+) Dendritic Cells in an Ovalbumin-Induced Airway Inflammation. Front Immunol 8, 718
33. Li, P., Yang, Q. Z., Wang, W., Zhang, G. Q., and Yang, J. (2018) Increased IL-4- and IL-17-producing CD8(+) cells are related to decreased CD39(+)CD4(+)Foxp3(+) cells in allergic asthma. J Asthma 55, 8-14
34. Zuska-Prot, M., Ziolkowski, H., Jaroszewski, J. J., and Maslanka, T. (2016) Distribution of CD4+CD8+ double positive T cells in a mouse model of allergic asthma. Pol J Vet Sci 19, 217-219
35. Schedel, M., Jia, Y., Michel, S., Takeda, K., Domenico, J., Joetham, A., Ning, F., Strand, M., Han, J., Wang, M., Lucas, J. J., Vogelberg, C., Kabesch, M., O'Connor, B. P., and Gelfand, E. W. (2016) 1,25D3 prevents CD8(+)Tc2 skewing and asthma development through VDR binding changes to the Cyp11a1 promoter. Nat Commun 7, 10213
36. Wang, A., Wang, Z., Cao, Y., Cheng, S., Chen, H., Bunjhoo, H., Xie, J., Wang, C., Xu, Y., and Xiong, W. (2015) CCL2/CCR2-dependent recruitment of Th17 cells but not Tc17 cells to the lung in a murine asthma model. Int Arch Allergy Immunol 166, 52-62
37. Jia, Y., Domenico, J., Takeda, K., Han, J., Wang, M., Armstrong, M., Reisdorph, N., O'Connor, B. P., Lucas, J. J., and Gelfand, E. W. (2013) Steroidogenic enzyme Cyp11a1 regulates Type 2 CD8+ T cell skewing in allergic lung disease. Proc Natl Acad Sci USA 110, 8152-8157
38. McNamara, J. T., Schramm, C. M., Singh, A., Secor, E. R., Jr., Guernsey, L. A., Lefrancois, L., and Thrall, R. S. (2012) Phenotypic changes to the endogenous antigen-specific CD8+ T cell response correlates with the development and resolution of allergic airway disease. Am J Pathol 180, 1991-2000

1. A method of treating or reducing the risk of asthma in a subject comprising providing to the subject an effective amount of fibroblasts and/or fibroblast-derived products.

2. The method of claim 1, wherein the method comprises providing to the subject an effective amount of fibroblasts.

3. The method of claim 2, wherein the fibroblasts are plastic-adherent and/or express CD105 and/or express CD73.

4-8. (canceled)

9. The method of claim 2, further comprising, prior to providing to the subject the effective amount of fibroblasts and/or fibroblast-derived products, exposing the fibroblasts to an effective amount of oxytocin.

10. (canceled)

11. The method of claim 1, wherein the method comprises providing to the subject an effective amount of fibroblast-derived products.

12. The method of claim 11, wherein the fibroblast-derived products comprise conditioned media derived from fibroblasts.

13. The method of claim 12, wherein the conditioned media stimulates proliferation of CD8+ cells in the subject.

14. The method of claim 12, wherein the conditioned media comprises FGF-1, FGF-2, and/or TGF-β from the fibroblasts.

15-19. (canceled)

20. The method of claim 11, wherein the fibroblast-derived products comprise microvesicles, apoptotic vesicles, nucleic acids, and/or exosomes from fibroblasts.

21. (canceled)

22. The method of claim 20, wherein the exosomes comprise CD81 and/or TNF-β.

23-26. (canceled)

27. The method of claim 1, wherein the method comprises reducing pulmonary inflammation in the subject.

28. The method of claim 27, wherein the pulmonary inflammation is associated with exposure to one or more allergens.

29. The method of claim 1, wherein the fibroblasts and/or fibroblast-derived products inhibit eosinophil activation, basophil activation, and/or mast cell activation in the subject.

30-31. (canceled)
